(19) United States
(12) Patent Application Publication
(10) Pub. No.: US 2009/0076290 A1
(43) Pub. Date: Mar. 19, 2009

(54) SELECTIVE OXIDATION OF TRITERPENES EMPLOYING TEMPO

(75) Inventors: Pavel A. Krasutsky, Duluth, MN (US); Alexei Pushechnikov, Kiev (UA); Tatiana Sergeeva, Los Angeles, CA (US)

Correspondence Address: SCHWEGMAN, LUNDBERG & WOESSNER, P.A., P.O. BOX 2938, MINNEAPOLIS, MN, 55402, US

(73) Assignee: REGENTS OF THE UNIVERSITY OF MINNESOTA, Minneapolis, MN (US)

(21) Appl. No.: 11/910,122

(22) PCT Filed: Mar. 29, 2006
(86) PCT No.: PCT/US2006/011791

§ 371 Date: Oct. 23, 2008

Related U.S. Application Data
(60) Provisional application No. 60/666,025, filed on Mar. 29, 2005.

(51) Int. Cl. C07J 53/00

(52) U.S. Cl. 552/510

(57) ABSTRACT

The present invention provides a process of preparing betulin-28-aldehyde from betulin. The process includes contacting betulin with a compound of formula (I), e.g., TEMPO (2,2,6,6-tetramethylpiperidine 1-oxyl) for a period of time effective to provide betulin-28-aldehyde. The present invention also provides a process of preparing betulinic acid. The process includes contacting betulin with a composition that includes: sodium hypochlorite (NaOCl); sodium chlorite (NaClO₂), potassium chlorite (KClO₂), or a combination thereof; and a compound of formula (I), e.g., TEMPO (2,2,6,6-tetramethylpiperidine 1-oxyl); for a period of time effective to provide betulinic acid.

### BACKGROUND OF THE INVENTION

Betulinic acid is useful as a potential therapeutic agent. For example, Pisha, E. et al., (1995) J. M. Nature Medicine, 1, 1046-1051 disclose that betulinic acid has antitumor activity against melanoma, e.g., MEL-1, MEL-2 and MEL4. In addition, Fujioka, T. et al., J. Nat. Prod., (1994) 57, 243-247 discloses that betulinic acid has anti-HIV activity in H9 lymphocytic cells.

Betulinic acid can be manufactured from betulin, which is present in large quantities in the outer birch bark of numerous species of birch trees. For example, a single paper mill in northern Minnesota generates nearly 30-70 tons of birch bark per day. Approximately 230,000 tons of birch bark are generated per year. Outer bark of Betula verrucosa (European commercial birth tree) contains nearly 25% betulin (Rainer Ekman, 1983, Horzforschung 37, 205-211). The outer bark of Betula paparifera (commercial birch of northern U.S. and Canada) contains nearly 5-18% betulin (see, U.S. patent Ser. No. 09/371,298). As such, vast quantities of betulin are available.

U.S. Pat. No. 5,804,575 issued to Pezzuto et al. discloses a five-step process for the synthesis of betulinic acid from betulin. Due to the length of time required to carry out this process and the yield it provides, it is not ideal for the commercial scale (e.g., kilogram) production of betulinic acid. Additionally, the process uses solvents and reagents that are hazardous and expensive, and the disclosed purification steps are not feasible on a commercial scale.

The first step in the preparation of betulinic acid from betulin-3-acetate was described by Ruzichka et al. (Helv. Chim. Acta., 21, 1706-1715 (1938)). The main obstacle for employing this method is the preparation of starting material (i.e., betulin-3-acetate). The selectivity of the hydrolysis of betulin-3,28-diacetate with potassium hydroxide provided about 60% betulin-3-acetate. The use of magnesium alcoholates (Yao-Chang Xu et al., J. Org. Chem., 61, 9086-9089 (1996)) in the selective deprotection of betulin-3,28-diacetate (Yao-Chang Xu et al., J. Org. Chem., 61, 9086-9089 (1996)) has several serious drawbacks. The selectivity of this process is about 81%. Additionally, the cost of magnesium alcoholates is fairly high. As such, this method is not attractive for the commercial scale production of betulinic acid.

The development of an industrially viable selective method for the conversion of primary alcohols to aldehydes is a very desirable target in synthetic organic chemistry, including the specific synthesis of betulin-28-aldehyde. U.S. Pat. No. 6,127,573 describes a method to oxidize primary alcohols to carboxylic acids with a TEMPO catalyst using CaClO₃ and NaClO. The primary alcohol can be a C₃-C₈ cycloalkyl (see, e.g., claim 1), but there is no disclosure or suggestion that the primary alcohol can be a polycyclic hydrocarbon having more than eight carbon atoms, e.g., a triterpenoid.

Zhao et al., Organic Synthesis, Vol. 81, p. 195 (2005), describe the oxidation of primary alcohols to carboxylic acids with sodium chlorite catalyzed by TEMPO and bleach. U.S. Pat. Nos. 6,407,270; 6,271,405; and 6,232,481 describe methods for manufacturing betulinic acid from betulin. U.S. Pat. Nos. 6,815,553; 6,634,575; and 6,392,070 describe methods for isolating betulinic acid from birch bark. However, there exists a need for additional methods for preparing betulinic acid and synthetic precursors thereof. Such methods should require relatively little time, should provide a relatively high overall yield, should be cost effective (i.e., should require relatively inexpensive reagents and solvents) relative to known procedures, and/or should satisfy the contemporary industrial demands from both safety and environmental points of view.

### SUMMARY OF THE INVENTION

The present invention provides a relatively cost effective, safe and efficient manner to convert a primary alcohol of a triterpenoid to the corresponding aldehyde. Specifically, the present invention provides a relatively cost effective, safe and efficient manner to convert betulin to betulin-28-aldehyde. The synthesis is a one-step method that typically affords up to about 90 wt. % aldehyde, and about 10 wt. % unreacted starting material. The oxidation employs a compound of formula (I), e.g., TEMPO (2,2,6,6-tetramethylpiperidine 1-oxyl). The crude aldehyde can be converted to the corresponding carboxylic acid (betulinic acid), where it can be separated from the unreacted starting material (e.g., betulin) employing, e.g., an acid-base washing.

The present invention provides a process of converting a primary alcohol of a triterpene, to the corresponding aldehyde. The present invention also provides a process of converting a primary alcohol of a triterpene, to the corresponding carboxylic acid. The processes employ a compound of formula (I), e.g., TEMPO (2,2,6,6-tetramethylpiperidine 1-oxyl), or an analogue thereof. Additionally, such conversions can be selectively carried out, such that any secondary hydroxyl (alcohol) groups present on the triterpene will not be oxidized.

The present invention also provides a process of preparing betulin-28-aldehyde from betulin. The process includes contacting betulin with a compound of formula (I), e.g., TEMPO (2,2,6,6-tetramethylpiperidine 1-oxyl) for a period of time effective to provide betulin-28-aldehyde.

The present invention also provides a process of preparing betulinic acid. The process includes contacting betulin with a composition that includes: sodium hypochlorite (NaOCl); sodium chlorite (NaClO₂), potassium chlorite (KClO₂), or a combination thereof; and a compound of formula (I), e.g., TEMPO (2,2,6,6-tetramethylpiperidine 1-oxyl); for a period of time effective to provide betulinic acid.

The present invention also provides a process of oxidizing a triterpene having a primary alcohol, to the corresponding triterpene having an aldehyde. The present invention also provides a process of subsequently oxidizing the triterpene having an aldehyde, to the corresponding triterpene having a carboxylic acid. The present invention also provides a process of oxidizing a triterpene having a primary alcohol, to the corresponding triterpene having a carboxylic acid. The triterpene having the primary alcohol optionally also includes a secondary alcohol, wherein the secondary alcohol is optionally protected with, e.g., an acyl group.

### DETAILED DESCRIPTION OF THE INVENTION

As used herein, the following terms and expressions have the indicated meanings. It will be appreciated that the compounds of the present invention contain asymmetrically substituted carbon atoms, and may be isolated in optically active or racemic forms. It is well known in the art how to prepare optically active forms, such as by resolution of racemic forms or by synthesis, from optically active starting materials. All chiral, diastereomeric, racemic forms and all geometric isomeric forms of a structure are intended, unless the specific stereochemistry or isomeric form is specifically indicated.

As used herein, “physiologically acceptable salts” refer to derivatives of the disclosed compounds wherein the parent compound is modified by making acid or base salts thereof. Examples of physiologically acceptable salts include, but are not limited to, mineral or organic acid salts of basic residues such as amines; alkali or organic salts of acidic residues such as carboxylic acids; and the like. The physiologically acceptable salts include the conventional non-toxic salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, isethionic, and the like.

The physiologically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

The phrase “physiologically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by and employed in the present invention.

“Substituted” is intended to indicate that one or more (e.g., 1, 2, 3, 4, or 5; preferably 1, 2, or 3; and more preferably 1 or 2) hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Alternatively, the suitable indicated groups can include, e.g., —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O⁻, —S(═O)₂OH, —S(═O)₂R, —OS(═O)₂OR, —S(═O)₂NR, —S(═O)R, —OP(═O)O₂RR, —P(═O)O₂RR—P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen: F, Cl, Br, or I; and each R is independently H, alkyl, aryl, heterocycle, protecting group or prodrug moiety. When a substituent is keto (i.e., ═O) or thioxo (i.e., ═S) group, then 2 hydrogens on the atom are replaced.

One diastereomer may display superior activity compared with the other. When required, separation of the racemic material can be achieved by HPLC using a chiral column or by a resolution using a resolving agent such as camphonic chloride as in Thomas J. Tucker, et al., J. Med. Chem. 1994 37, 2437-2444. A chiral compound may also be directly synthesized using a chiral catalyst or a chiral ligand, e.g. Mark A. Huffman, et al., J. Org. Chem. 1995, 60, 1590-1594.

The term “alkyl” refers to a monoradical branched or unbranched saturated hydrocarbon chain preferably having from 1 to 10 carbon atoms, preferably 1 to 6 carbon atoms, and more preferably from 1 to 4 carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (1-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃. The alkyl can be unsubstituted or substituted.

The term “alkenyl” refers to a monoradical branched or unbranched partially unsaturated hydrocarbon chain (i.e. a carbon-carbon, sp² double bond) preferably having from 2 to 10 carbon atoms, preferably 2 to 6 carbon atoms, and more preferably from 2 to 4 carbon atoms. Examples include, but are not limited to, ethylene or vinyl (—CH═CH₂), allyl (—CH₂CH═CH₂), cyclopentenyl (—C₅H₇), and 5-hexenyl (—CH₂ CH₂CH₂CH₂CH═CH₂). The alkenyl can be unsubstituted or substituted.

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like. The alkoxy can be unsubstituted or substituted.

The term “aryl” refers to an unsaturated aromatic carbocyclic group of from 6 to 12 carbon atoms having a single ring (e.g., phenyl) or multiple condensed (fused) rings, wherein at least one ring is aromatic (e.g., naphthyl, dihydrophenanthrenyl, fluorenyl, or anthryl). The aryl can be unsubstituted or substituted.

The term “cycloalkyl” refers to cyclic alkyl groups of from 3 to 10 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like. The cycloalkyl can be unsubstituted or substituted.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, selected from alkyl, alkenyl, alkynyl, alkoxy, halo, haloalkyl, hydroxy, hydroxyalkyl, aryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl and cyano. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

“Heterocycle” as used herein includes by way of example and not limitation those heterocycles described in Paquette, Leo A.; Principles of Modern Heterocyclic Chemistry (W.A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, or S).

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl:

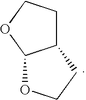

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” refers to a saturated, unsaturated or aromatic ring having 3 to 7 carbon atoms as a monocycle, 7 to 12 carbon atoms as a bicycle, and up to about 30 carbon atoms as a polycycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl.

The term “alkanoyl” refers to C(═O)R, wherein R is an alkyl group as previously defined.

The term “alkoxycarbonyl” refers to C(—O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “nitro” refers to —NO₂.

The term “trifluoromethyl” refers to —CF₃.

The term “trifluoromethoxy” refers to —OCF₃.

The term “cyano” refers to —CN.

The term “hydroxy” refers to —OH.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

As used herein, “aldehyde” refers to the functional group —C(═O)H, or any compound that includes such a group.

As used herein, “primary alcohol” refers to a hydroxyl group that is directly bonded to a carbon atom, wherein that carbon atom is directly bonded to exactly one other carbon atom. The term also refers to those compounds that include such a group (i.e., a hydroxyl group that is directly bonded to a carbon atom, wherein that carbon atom is directly bonded to exactly one other carbon atom).

As used herein, “secondary alcohol” refers to a hydroxyl group that is directly bonded to a carbon atom, wherein that carbon atom is directly bonded to exactly two other carbon atoms. The term also refers to those compounds that include such a group (i.e., a hydroxyl group that is directly bonded to a carbon atom, wherein that carbon atom is directly bonded to exactly two other carbon atoms).

As used herein, “nitroxyl radical” refers to functional group (N—O.) and to compounds that include such a group.

As used herein, “contacting” refers to the act of touching, making contact, or of immediate proximity, including at the molecular level.

As used herein, “triterpene” or “triterpenoid” refers to a plant secondary metabolite that includes a hydrocarbon, or its oxygenated analog, that is derived from squalene by a sequence of straightforward cyclizations, functionalizations, and sometimes rearrangement. Triterpenes or analogues thereof can be prepared by methods known in the art, i.e., using conventional synthetic techniques or by isolation from plants. Suitable exemplary triterpenes and the biological synthesis of the same are disclosed, e.g., in R. B. Herbert, The Biosynthesis of Secondary Plant Metabolites, 2nd. ed. (London: Chapman 1989). The term “triterpene” refers to one of a class of compounds having approximately 30 carbon atoms and synthesized from six isoprene units in plants and other organisms. Triterpenes consist of carbon, hydrogen, and optionally oxygen. Most triterpenes are secondary metabolites in plants. Most, but not all, triterpenes are pentacyclic. Examples of triterpenes include betulin, allobetulin, lupeol, friedelin, and all sterols, including lanosterol, stigmasterol, cholesterol, β-sitosterol, and ergosterol. Additional examples of triterpenes include those described, e.g., in Published U.S. Patent Application Nos. 2004/0097436, 2002/0128210, and 2002/0119935.

The triterpene having a primary alcohol can be, for example:

a triterpene from the fusidane-lanostane group (e.g., poricoic acid F, Cycloart-24-ene-3β,16β,21-triol; genin, lavenone, dammar-25-ene-3b, 20,21,24-tetrol; or dichapetalin A);

a triterpene from the lupane group (e.g., betulin, betulone, lup-2α,3β,28-triol; or lup-20(29)-ene-3β,23-diol);

a triterpene from the oleanane group (e.g., 28-hydroxyoleanan-13 (18)-en-3-one; oleana-11,13(18)-diene-3β,16β,23,28-pentol; olean-12-ene-3b,11α,16β,21α,23,28-hexyl; wilforol C; bridgesigenin C, alamosenogedin, saikaogenin Q, mimusopgenone, mimugenone 191, 3b,22b,24-trihydroxyoleanane-12-ene-28-al; 3-coumaroyllarjunolic acid; vacogenin, 29,30-dinorolcanane-13β,15β,16β,26-tetrol; bellisonic acid 204; anemoclemoside A, phachanol A, phachanol B; or phachanol C);

a triterpene from the ursane group (e.g., grahamidiol); or

a miscellaneous triterpene (e.g., pochanol or trevoagenin A).

As used herein, “betulin” refers to 3β,28-dihydroxy-lup-20(29)-ene. Betulin is a pentacyclic triterpenoid derived from the outer bark of paper birch trees (Betula papyrifera, B. pendula, B. verucosa, etc.). The CAS Registry No. is 473-98-3. It can be present at concentrations of up to about 24% of the bark of white birch. Merck Index, twelfth edition, page 1236 (1996). Structurally, betulin is shown below:

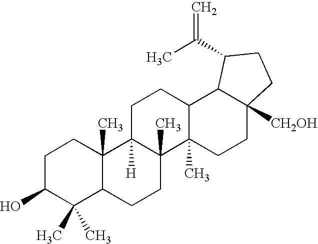

As used herein, “betulinic acid” refers to 3(β)-hydroxy-20(29)-lupaene-28-oic acid; 9-hydroxy-1-isopropenyl-5a,5b,8,8,11a-pentamethyl-eicosahydro-cyclopenta[a]chrysene-3a-carboxylic acid. The CAS Registry No. is 472-15-1. Structurally, betulinic acid is shown below:

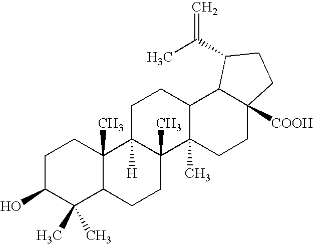

As used herein, “betulin aldehyde” refers to 3(β)-hydroxy-lup-20(29)-en-28-al; Lup-20(29)-en-28-al, 3β-hydroxy-(8CI); Lup-20(30)-en-28-al, 3β-hydroxy-(7CI); 3aH-Cyclopenta[a]chrysene, lup-20(29)-en-28-al deriv.; Betulinaldehyde; Betulinic aldehyde; or Betunal. The CAS Registry Number is 13159-28-9. Structurally, betulin aldehyde is shown below:

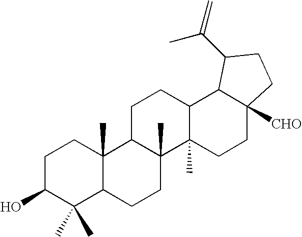

As used herein, “carboxy” refers to —C(═O)O or —COOH.

As used herein, “TEMPO” refers to 2,2,6,6-tetramethylpiperidine 1-oxyl, having the CAS Registry No. of 2564-83-2, which is a compound of the formula

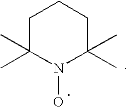

As used herein, “4-(2-Chloroacetamido)-TEMPO” refers to 4-(2-chloroacetamido)-2,2,6,6-tetramethyl-1-piperidinyloxy, free radical, which is a compound of the formula

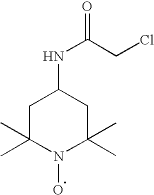

As used herein, “TEMPO, polymer-bound” refers to having a particle size 100-200 mesh, loading: 1.0 mmol/g, 1% cross-linked with divinylbenzene; which is a compound of the formula

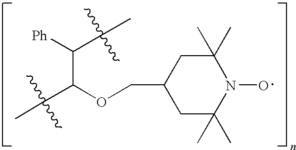

wherein the wavy lines indicate bonds of the polystyrene monomeric units to adjacent monomeric units.

As used herein, “4-(2-Bromoacetamido)-TEMPO” refers to 4-(2-bromoacetamido)-2,2,6,6-tetramethyl-1-piperidinyloxy, free radical; which is a compound of the formula

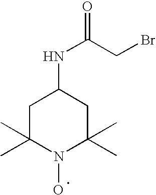

### The CAS Registry Number is 24567-97-3.

As used herein, “4-(2-iodoacetamido)-TEMPO” refers to 4-(2-iodoacetamido)-2,2,6,6-tetramethylpiperidine 1-oxyl or 4-(2-iodoacetamido)-2,2,6,6-tetramethyl-1-piperidinyloxy, free radical; which is a compound of the formula

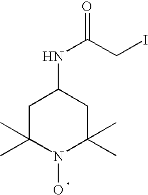

### The CAS Registry Number is 25713-24-0.

As used herein, “4-cyano-TEMPO” refers to a compound of the formula

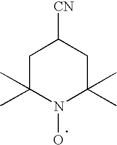

### The CAS Registry Number is 38078-71-6.

As used herein, “4-maleimido-TEMPO” refers to 4-maleimido-2,2,6,6-tetramethyl-1-piperidinyloxy, free radical; which is a compound of the formula

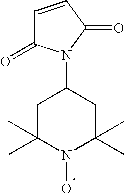

### The CAS Registry Number is 15178-63-9.

As used herein, “4-methoxy-TEMPO” refers to 4-methoxy-2,2,6,6-tetramethyl-1-piperidinyloxy, free radical; which is a compound of the formula

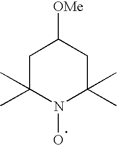

### The CAS Registry Number is 95407-69-5.

As used herein, “4-oxo-TEMPO” refers to 4-Oxo-2,2,6,6-tetramethyl-1-piperidinyloxy, free radical; which is a compound of the formula

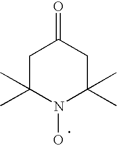

### The CAS Registry Number is 2896-70-0.

As used herein, “TEMPO on silica gel” refers to 2,2,6,6-Tetramethyl-1-piperinyloxy, free radical on silica gel; which is a compound of the formula

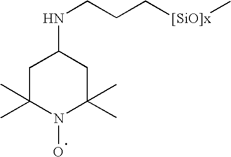

As used herein, “4-amino-2,2,6,6-tetramethylpiperidine-1-oxyl” refers to 4-Amino-2,2,6,6-tetramethylpiperidine-1-oxyl; 4-Amino-2,2,6,6-tetramethylpiperidinyloxy, free radical; or 4-Amino-TEMPO; which is a compound of the formula

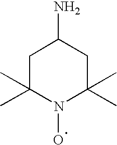

### The CAS Registry Number is 14691-88-4.

As used herein, “4-carboxy-2,2,6,6-tetramethylpiperidine 1-oxyl” refers to 4-carboxy-2,2,6,6-tetramethylpiperidine 1-oxyl; 4-carboxy-TEMPO; or 4-carboxy-2,2,6,6-tetramethylpiperidinyloxy, free radical; which is a compound of the formula

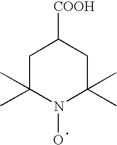

### The CAS Registry Number is 37149-18-1.

As used herein, “4-acetamido-2,2,6,6-tetramethylpiperidine 1-oxyl” refers to 4-Acetamido-2,2,6,6-tetramethylpiperidine 1-oxyl; which is a compound of the formula

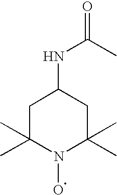

### The CAS Registry Number is 14691-89-5.

As used herein, “4-(2-chloroacetamido)-2,2,6,6-tetramethylpiperidine 1-oxyl” refers to 4-(2-Chloracetamido)-TEMPO; which is a compound of the formula.

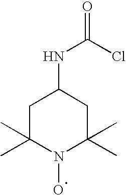

### The CAS Registry Number is 36775-23-2.

As used herein, “TEMPOL” refers to 4-hydroxy-TEMPO or 4-hydroxyl-2,2,6,6-tetramethylpiperidine-1-oxyl; which is a compound of the formula:

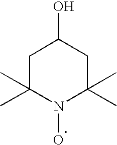

It is appreciated that those of skill in synthetic organic chemistry understand that starting materials, reagents, solvents and catalysts are typically characterized by their chemical name or structure, as they are introduced into a chemical reaction. While these compounds may undergo a substantial conversion prior to or during a specified reaction step, reference to these compounds is acceptable and appropriate to those of skill in synthetic organic chemistry. For example, TEMPO may be converted, in situ, to the corresponding N-oxoammonium ion, which oxidizes the primary alcohol (betulin) to the aldehyde (betulin-28-aldehyde), such that TEMPO itself does not, but the corresponding N-oxoammonium ion, contacts or oxidizes the primary alcohol (betulin). Reference to TEMPO contacting the primary alcohol (betulin) and/or reference to TEMPO oxidizing the primary alcohol (betulin), however, is acceptable and appropriate to those of skill in synthetic organic chemistry. As such, as used herein, “TEMPO” also includes, e.g., the corresponding N-oxoammonium ion as well as the corresponding hydroxylamine. This also applies to the compound of formula (I), e.g., the following compounds: 4-(2-Chloroacetamido)-TEMPO; TEMPO, polymer-bound; 4-(2-Bromoacetamido)-TEMPO; 4-(2-iodoacetamido)-TEMPO; 4-cyano-TEMPO; 4-maleimido-TEMPO; 4-methoxy-TEMPO; 4-oxo-TEMPO; TEMPO on silica gel; 4-amino-2,2,6,6-tetramethylpiperidine-1-oxyl; 4-carboxy-2,2,6,6-tetramethylpiperidine 1-oxyl; 4-acetamido-2,2,6,6-tetramethylpiperidine 1-oxyl; 4-(2-chloroacetamido)-2,2,6,6-tetramethylpiperidine 1-oxyl; and TEMPOL.

As used herein, “oxoammonium ion” or “N-oxoammonium ion” refers to the functional group (N⁺═O), and to compounds that include such a group. See, e.g., M. F. Semmelhack et al., J. Am. Chem. Soc., 1983, 105, 4492-4494.

As used herein, “nitroxyl radical” refers to functional group (N—O.) and to compounds that include such a group.

As used herein, “NaClO₂” refers to sodium chlorite.

As used herein, “KClO₂” refers to potassium chlorite.

As used herein, “selectively oxidized” refers to a functional group (e.g., primary alcohol) of a compound undergoing a chemical conversion (e.g., to an aldehyde) and another functional group (secondary alcohol) of the same compound undergoing a chemical conversion (e.g., to a ketone), such that the ratio of the two functional group transformations is least about 90:10, at least about 95:5, or at least about 99:1, respectively. Alternatively, the term refers to a functional group (e.g., primary alcohol) of a compound undergoing a chemical conversion (e.g., to an aldehyde) and that same functional group (primary alcohol) undergoing a separate chemical conversion (e.g., to a carboxylic acid), such that the ratio of the two functional group transformations is least about 90:10, at least about 95:5, or at least about 99:1, respectively.

As used herein, “selectively converted” refers to a functional group (e.g., primary alcohol) of a compound being oxidized (e.g., to an aldehyde) and another functional group (secondary alcohol) of the same compound undergoing a chemical conversion (e.g., to a ketone), such that the ratio of the two functional group transformations is least about 90:10, at least about 95:5, or at least about 99:1, respectively. Alternatively, the term refers to a functional group (e.g., primary alcohol) of a compound being oxidized (e.g., to an aldehyde) and the same functional group (primary alcohol) undergoing a separate chemical conversion (e.g., to a carboxylic acid), such that the ratio of the two functional group transformations is least about 90:10, at least about 95:5, or at least about 99:1, respectively.

As used herein, “separating” refers to the process of removing solids from a mixture. The process can employ any technique known to those of skill in the art, e.g., decanting the mixture, filtering the solids from the mixture, or a combination thereof.

Without being limited to any particular theory, it is believed that the oxidation of betulin to betulin-28-aldehyde and/or betulinic acid with TEMPO follows the mechanism as shown in Scheme 1 below (see, Organic Synthesis, Vol. 81, p. 195 (2005) and references cited therein). In that mechanism, TEMPO radical is first oxidized by NaOCl to the N-oxoammonium ion, which rapidly oxidizes the primary alcohol (betulin) to the aldehyde (betulin-28-aldehyde) and gives a molecule of the hydroxylamine. The aldehyde is then oxidized by NaOCl₂ to the carboxylic acid (betulinic acid) and regenerates a molecule of NaOCl. The hydroxylamine can either be directly oxidized or can undergo a syn proportionation to give two molecules of TEMPO radical. Although the exact mechanism of TEMPO-catalyzed oxidation of alcohols is still unclear, previous work has shown that N-oxoammonium ion and hydroxylamine are involved.

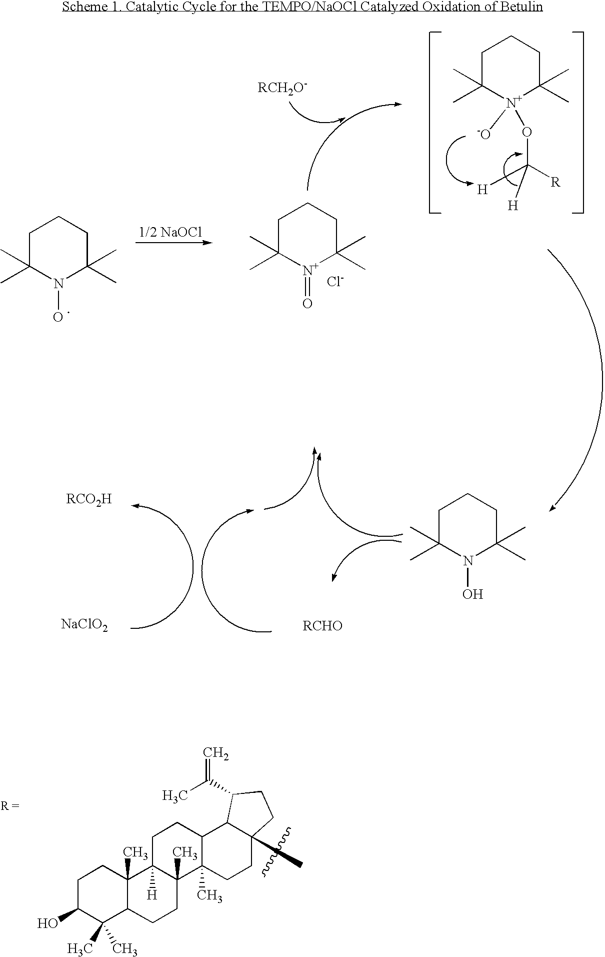

### EXAMPLES

### Example 1

### Preparation of Betulinic Aldehyde

Betulin (0.2 g) was loaded into a round bottom flask together with TEMPO (25 mg), CH₂Cl₂ (3 mL), aqueous KH2PO4 solution (7 g/L, 3 mL), and t-BuOH (3 mL). 5 mL of oxidizing solution (5 mL of diluted bleach (bleach/water=1/1) mixed with 5 mL of aq. NaClO2 (4.57 g NaClO2/20 mL H2O)) was added with vigorous stirring. The mixture was stirred at room temperature overnight, organic and aqueous layers were separated. The aqueous layer was extracted with CH₂Cl₂ (3 times), the combined organic solution was washed with water 3 times and dried over anhydrous sodium sulfate. 60% yield of betulinic aldehyde was achieved after solvent removing and purification on silica (hexane/ether 1/1).

### Example 2

### Preparation of Betulinic Acid

Betulinic aldehyde (0.73 g) was placed in a mixture of t-butyl alcohol (5 mL) and 2-methyl-2-butene (4 mL). The mixture was vigorously stirred until complete dissolution. The apparatus was places into a bowl, with ice and water (1/1). Then, a freshly prepared solution of sodium chlorite (NaClO₂, 0.45 g in 4 mL of water) and dihydrogen potassium phosphate (0.68 g in 6 mL of water) were added at 20° C. during 0.5 hour under efficient stirring. Stirring at 20° C. was continued for 4 hours. Then the precipitate was filtered and washed 2× with 5 mL water and 5 mL of ethanol. Dichloromethane (8 mL) and water (7 mL) were added to the organic solution, shaken in a separatory funnel and the organic layer was separated. Betulinic acid (0.49 g, purity 96%+) was obtained.

### Example 3

### Preparation of Betulinic Aldehyde 3-acetate

Betulin-3-acetate (0.4 g) was loaded into a round-bottom flask together with TEMPO (50 mg), CH₂Cl₂ (6 mL), aqueous KH₂PO₄ solution (7 g/L, 6 mL), and t-BuOH (6 mL). 10 mL of oxidizing solution (10 mL of diluted bleach (bleach/water=1/1) mixed with 10 mL of aq. NaClO₂ (4.57 g NaClO₂/20 mL H2O)) was added with vigorous stirring. The mixture was stirred at room temperature overnight, then organic and aqueous layers were separated. The aqueous layer was extracted with CH₂Cl₂ (3 times), the combined organic solution was washed with water 3 times and dried over anhydrous sodium sulfate. A 70% yield of betulinic aldehyde was achieved after solvent removal and purification on silica (hexane/ether 1/1).

### Example 4

### Preparation of betulin-28-aldehyde

To a 125 mL, 3-necked, round-bottomed flask was charged 0.1765 g of TEMPO (1.13 mmol, 1 equiv), 0.095 g of NaHCO₃ (1.13 mmol, 1 equiv), 0.1162 g of NaBr (1.13 mmol, 1 equiv), and 5 mL of H₂O. The resulting mixture was stirred at rt for about 5 min, and then to the reaction mixture was added 0.5 g (1.13 mmol) of betulin and 50 mL of CH₂Cl₂. The batch was stirred at rt for about 10 min, and then was placed in an ice bath. To the reaction vessel was then added 20 mL of a 0.5% NaClO solution (1.34 mmol, 1.19 equiv) through a syringe pump (rate=0.1 mL/min, addition time=3 h 20 min) at 1° C. (internal temperature), and the resultant mixture was stirred at 1° C. for another 30 min. HPLC analysis of the batch showed ca. 97% of betulinic aldehyde, 1.2% of the starting material, and 1.7% of betulonic aldehyde. To the batch was then added 10 mL of 0.5% Na₂SO₃ solution while maintaining the internal temperature below 3° C., and the mix was stirred at <3° C. for another 10 min. The layers in the mix were separated, and the aqueous layer was extracted with 20 mL of CH₂Cl₂. The combined organic phase was dried (Na₂SO₄), filtered, and concentrated under reduced pressure to afford 0.718 g of an orange foam that contained ca. 97% of the desired product with ca. 0.5% of betulin and 1.5% of betulonic aldehyde as determined by HPLC. The crude material was then dissolved in 3 mL of CH₂Cl₂, and then was loaded onto a column containing 25 g of silica gel (pre-packed with hexanes). The column was then eluted with 200 mL of 5% EtOAc in hexanes, and the eluent was collected in 1×100 mL and 3×30 mL cuts. The column was then eluted with 200 mL of 20% EtOAc in hexanes, and the eluent was collected in 30 mL fractions. Fractions 5 to 7 were combined (TLC analyzed), and solvent was removed under reduced pressure to afford 0.487 g of a white solid. HPLC analysis of the product showed the desired betulinic aldehyde at ca. 98% purity with ca. 1.6% of betulonic aldehyde. Yield was 96%.

### Example 5

### Preparation of Betulinic Acid

Stirring a 5-mL CH₂Cl₂ solution containing 0.5 g of betulinic aldehyde (1.13 mmol) with 5-mL of aqueous solution containing 0.189 g of NaClO₂ (80%, 1.13 mmol, 1 equiv) and 0.153 g of KH₂PO₄ (1.13 mmol, 1 equiv) at 25° C. (oil bath temperature) over night, produced 9% of betulinic acid with ca. 89% of the starting material by HPLC. After doubling CH₂Cl₂ and H₂O volume, heating the reaction mixture at 40° C. (oil bath temperature), and the addition of another 1 g of NaClO₂ (8.85 mmol, 7.8 equiv), ca. 26% of the desired acid was found with 62% of betulinic aldehyde by HPLC. To the batch was then added 2.5 mL of THF to help dissolve the solid formed during the reaction, and the resultant mixture (two clear phases) was heated in an oil bath at 40° C. over night. HPLC analysis of the batch showed no starting material as well as the 72% of the desired product. The layers were cut, and the aqueous layer was extracted with another 10 mL of CH₂Cl₂. Emulsion during the course of work-up was observed, and was dealt with best judgment. The combined organic layer was dried (Na₂SO₄), filtered, and concentrated under reduced pressure to afford 0.402 g of a white solid. HPLC analysis of the crude showed the desired betulinic acid at ca. 72% pure.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of preparing betulin-28-aldehyde from betulin, the method comprising contacting betulin with a compound of formula (I): 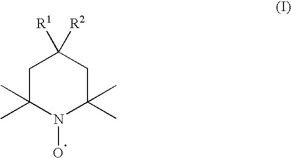
wherein,
each of R¹ and R² is independently hydrogen, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, carboxyl, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyannato, sulfamoyl, sulfinamoyl, sulfino, sulfo, sulfoamino, thiosulfo, NR^{x}R^{y} or COOR^{x}, wherein each R^{x} and R^{y} are independently H, alkyl, alkenyl, aryl, heteroaryl, heterocycle, cycloalkyl or hydroxyl; wherein any alkyl group is optionally substituted on carbon with keto (═O); wherein any alkyl group is optionally interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO), sulfonyl (SO₂) or [SiO]x, wherein x is about 1-10,000; or R¹ and R² together are thioxo (═S) or keto (═O);
for a period of time, effective to provide betulin-28-aldehyde.

2. The method of claim 1, wherein R¹ is hydrogen and R² is hydrogen, alkyl, alkoxy, haloalkyl, hydroxy, heterocycle, amino, alkylamino, cyano, carboxyl; wherein any alkyl group is optionally substituted on carbon with keto (═O); wherein any alkyl group is optionally interrupted with one or more imino (—N(H)—), carbonyl (—C(═O)—) or [SiO]x, wherein x is about 1-10,000; or R¹ and R² together are keto (═O).

3. (canceled)

4. The method of claim 1, wherein the compound of formula (I) is TEMPO (2,2,6,6-tetramethylpiperidine 1-oxyl).

5. The method of claim 1, wherein at least about 10 kg of betulin is employed.

6-7. (canceled)

8. The method of claim 1, wherein the contacting occurs in the presence of a biphasic solvent system that comprises at least one polar aprotic solvent and at least one polar protic solvent.

9-10. (canceled)

11. The method of claim 1, wherein the contacting occurs in the presence of a biphasic solvent system comprising a phase transfer catalyst comprising at least one alcohol.

12-14. (canceled)

15. The method of claim 1, wherein the pH of the composition is maintained between about 4.0 and 10.0.

16. (canceled)

17. The method of claim 1, wherein the composition further comprises a buffer, sufficient to maintain the pH of the composition to between about 4.0 to about 10.0.

18-19. (canceled)

20. The method of claim 1, wherein the contacting occurs at a temperature of about 25° C. to about 75° C. for up to about 24 hours.

21-22. (canceled)

23. The method of claim 1, wherein the betulin-28-aldehyde is obtained in at least about 65 mole percent, relative to the betulin.

24. (canceled)

25. The method of claim 1, further comprising converting betulin-28-aldehyde into betulinic acid employing NaClO₂, KClO₂, or a combination thereof.

26-31. (canceled)

32. A method of preparing betulinic acid, the method comprising contacting betulin with a composition comprising:
sodium hypochlorite (NaOCl);
sodium chlorite (NaClO₂), potassium chlorite (KClO₂), or a combination thereof; and
a compound of formula (I): 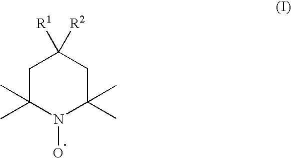
wherein,
each of R¹ and R² is independently hydrogen, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, carboxyl, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyannato, sulfamoyl, sulfinamoyl, sulfino, sulfo, sulfoamino, thiosulfo, NR^{x}R^{y} or COOR^{x}, wherein each R^{x} and R^{y} are independently H, alkyl, alkenyl, aryl, heteroaryl, heterocycle, cycloalkyl or hydroxyl; wherein any alkyl group is optionally substituted on carbon with keto (═O); wherein any alkyl group is optionally interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO), sulfonyl (SO₂) or [SiO]x, wherein x is about 1-10,000; or R¹ and R² together are thioxo (═S) or keto (═O);
for a period of time effective to provide betulinic acid.

33. The method of claim 32, wherein R¹ is hydrogen and R² is hydrogen, alkyl, alkoxy, haloalkyl, hydroxy, heterocycle, amino, alkylamino, cyano, carboxyl; wherein any alkyl group is optionally substituted on carbon with keto (═O); wherein any alkyl group is optionally interrupted with one or more imino (—N(H)—), carbonyl (—C(═O)—) or [SiO]x, wherein x is about 1-10,000, or R¹ and R² together are keto (═O).

34. (canceled)

35. The method of claim 32, wherein the compound of formula (I) is TEMPO (2,2,6,6-tetramethylpiperidine 1-oxyl).

36. The method of claim 32, wherein at least about 10 kg of betulin is employed.

37-38. (canceled)

39. The method of claim 32, wherein the contacting occurs in the presence of a biphasic solvent system that comprises at least one polar aprotic solvent and at least one polar protic solvent.

40-41. (canceled)

42. The method of claim 32, wherein the contacting occurs in the presence of a biphasic solvent system comprising a phase transfer catalyst comprising at least one alcohol.

43-45. (canceled)

46. The method of claim 32, wherein the pH of the composition is maintained between about 4.0 and 10.0.

47. (canceled)

48. The method of claim 32, wherein the composition further comprises a buffer, sufficient to maintain the pH of the composition to between about 4.0 to about 10.0.

49-50. (canceled)

51. The method of claim 32, wherein the contacting occurs at a temperature of about 25° C. to about 75° C. for up to about 24 hours.

52-59. (canceled)
